(19) United States
(12) Patent Application Publication
(10) Pub. No.: US 2005/0027262 A1
(43) Pub. Date: Feb. 3, 2005

(54) Endovascular tumescent infusion apparatus and method

(76) Inventors: William M. Appling, Granville, NY (US); Robert Torrance Andrews, Mercer Island, WA (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/909,033

(22) Filed: Jul. 30, 2004

Related U.S. Application Data
(60) Provisional application No. 60/491,573, filed on Jul. 31, 2003.

(51) Int. Cl. A61M 5/178

(52) U.S. Cl. 604/246

(57) ABSTRACT

A tumescent fluid infusion apparatus for use in treatment of a vascular disease includes a needle attached to a valve device. The valve device is designed to receive tumescent fluid from a fluid source. When the valve device is in an open position, it administers the tumescent fluid from the fluid source through the needle channel to allow a single hand of a user to control the needle insertion and the infusion of the tumescent fluid to free the other hand for operating a probe such as an ultrasound probe to eliminate the requirement of a second operator. The infusion apparatus also allows a user to control the amount of infused fluid without requiring syringe changes.

### CROSS REFERENCE TO RELATED APPLICATIONS

This application claims priority to U.S. provisional application no. 60/491,573, filed Jul. 31, 2003, which is incorporated herein by reference.

### FIELD OF THE INVENTION

The present invention relates to a medical device and method for treatment of blood vessels. More particularly, the present invention relates to an endovascular tumescent infusion apparatus and method for minimally invasive treatment of venous reflux disease.

### BACKGROUND OF THE INVENTION

Veins can be broadly divided into three categories: the deep veins, which are the primary conduit for blood return to the heart; the superficial veins, which parallel the deep veins and function as a channel for blood passing from superficial structures to the deep system; and topical or cutaneous veins, which carry blood from the end organs (e.g., skin) to the superficial system. Veins are thin-walled and contain one-way valves that control blood flow. Normally, the valves open to allow blood to flow into the deep veins and close to prevent back-flow into the superficial veins. When the valves are malfunctioning or only partially functioning, however, they no longer prevent the back-flow of blood into the superficial veins. This condition is called reflux. As a result of reflux, venous pressure builds within the superficial system. This pressure is transmitted to topical veins, which, because the veins are thin walled and not able to withstand the increased pressure, become dilated, tortuous or engorged.

In particular, venous reflux in the lower extremities is one of the most common medical conditions of the adult population. It is estimated that venous reflux disease affects approximately 25% of adult females and 10% of males. Symptoms of reflux include varicose veins and other cosmetic deformities, as well as aching, itching, and swelling of the legs. If left untreated, venous reflux may cause severe medical complications such as bleeding, phlebitis, ulcerations, thrombi and lipodermatosclerosis.

Endovascular thermal therapy is a relatively new treatment technique for venous reflux diseases. With this technique, thermal energy generated by laser, radio or microwave frequencies is delivered to the inner vein wall causing vessel ablation or occlusion. Typically a catheter, fiber or other delivery system is percutaneously inserted into the lumen of the diseased vein. Thermal energy is delivered from the distal end of the delivery system as the device is slowly withdrawn through the vein.

The procedure begins with an introducer catheter or sheath being placed into the main superficial vein, called the saphenous vein, at a distal location and advanced to within a few centimeters of point at which the saphenous vein enters the deep vein system, (the sapheno-femoral junction). Once the sheath is properly positioned and after the instillation of tumescent anesthesia as described below, the thermal delivery system is inserted into the lumen of the sheath and advanced until positioned near the saphenous-femoral junction area. It is to be noted, however, that in many vascular treatment procedures, the sheath is optional. To treat the vein, the energy source is activated causing energy to be emitted from the distal end of the thermal delivery system into the vessel. The energy reacts with the vessel wall causing cell necrosis and eventual vein collapse. With the energy source turned on, the delivery device is slowly withdrawn until the entire diseased segment of the vessel has been treated.

Prior to the application of thermal energy, tumescent anesthesia is injected along the entire length of the vein into space between the vein and the surrounding perivenous tissue. A mixture of saline and 0.1-0.5% lidocaine or other similar anesthetic agent is typically used. Tumescent anesthesia or tumescent fluid as used herein is a fluid that anatomically isolates the saphenous vein that creates a barrier to protect the tissue and nerves from the thermal energy, and/or reduces patient pain during the procedure.

The tumescent injections are typically administered every few centimeters along the entire length of the vein under ultrasonic guidance. A 10 or 20 cc syringe is filled with the solution and then attached to a micropuncture needle or flexible tube set. Relatively small syringes are used in order to generate sufficient pressure during the injection to cause the fluid to travel longitudinally along the perivenous space. Small syringes are also preferred because they can be more easily handled by a treating physician than larger, bulky syringes. The length of the vein being treated varies but is typically between 30 and 50 cm long. A total of approximately 60-120 cc of fluid is generally injected along the length of the vein during a normal treatment procedure.

Tumescent injections are delivered under ultrasound guidance. Ultrasound is used to visualize the vein, confirm proper location of the needle tip in the perivenous space, and to determine correct injection volumes. Ultrasound images are obtained by use of a handheld transducer that must be held in a precise position relative to the anatomy being visualized. After the user has confirmed that the needle tip is correctly positioned between the vein and fascia tissue through ultrasonic imaging, the tumescent fluid is slowly injected. Under ultrasound, the physician can see the fluid being injected and filling the perivenous space. The fluid eventually begins to dissipate radially into the surrounding tissue based on distance, resistance and venous anatomy. At this point, the physician removes the needle and repositions it to another location for the next injection.

One problem with the current method of tumescent injections is the difficulty in controlling the needle, syringe and ultrasonic transducer or probe all at the same time. As illustrated in FIG. 1, the physician utilizes the syringe as a handle to control the needle position and injection volumes. The physician must maintain the needle device 5 position while holding the syringe 30 and depressing the plunger to inject the tumescent agent. In addition, the ultrasound probe 19 must be held in position over the injection area in order to visualize the needle tip placement and the fluid flow path. Maintaining the position of the needle device 5 while depressing the syringe 30 plunger or otherwise adjusting the syringe is difficult since the two components are directly connected. Optimal control over the needle position occurs when the operator's hand is holding the needle hub. The further away the operator's hand is from the needle the more difficult it is to control needle placement. When injecting fluid using the method depicted in FIG. 1, the operator's hand holds the proximal section of syringe so as to control the plunger. This hand position negatively impacts the accuracy and control over the needle position. The bulkiness of the syringe and the decreased control over the needle makes it difficult for the physician to operate the syringe 30 without impacting the position of the needle device 5.

The problem of inadvertent needle movement after initial positioning has been addressed by using a needle with a flexible tubing set as shown in FIG. 2. With this technique, flexible tubing 31 placed between the needle device 5 and the syringe 30 allows for independent movement and adjustments of the syringe 30 without causing a corresponding movement of the needle device 5. Control and accuracy during injection is increased because the syringe can be operated independently of the needle. As shown in FIG. 2, however, this technique requires two operators to perform the procedure. One operator guides the ultrasound probe and controls the needle position while the other one controls syringe and injection volumes. Although this method increases control, it is expensive, inefficient and requires coordination between the two operators. Both operators are performing within the sterile field, and accordingly must be scrubbed and prepped for sterile conditions.

Another problem with the current procedure involves the risk of introducing air into the body during syringe changes. As previously discussed, 10 or 20 cc syringes are used to ensure that sufficient pressure can be generated during the injection. Use of the relatively small syringe means that the operator needs to change syringes numerous times during the procedure. When the fluid level in the syringe becomes low, the operator must disconnect the syringe from the needle and replace it with another, pre-filled syringe. During the syringe exchange, a small amount of air often enters the needle through the hub opening. Subsequent injection of the tumescent agent then cause the air pocket to advance through the needle into the surrounding tissue.

Although a small amount of air introduced into the tissue will not harm the patient, the air creates an ultrasonic shadow that impairs the operator's imaging visibility. Specifically, the air bubble creates a void on the ultrasound display. Any anatomical structure behind the air bubble may be obscured from view, including the vein and needle tip.

Multiple syringe change outs are also very time consuming, increasing overall procedure time and costs. Typically, the multiple syringes are filled with the lidocaine/saline solution prior to the procedure. The procedure preparation time is lengthened by the need for multiple syringes. The actual procedure time is also lengthened by the requirement for syringe exchanges during injections. In addition, multiple syringes increase the overall cost of the procedure.

Therefore, it is desirable to provide a tumescent fluid infusion device that is efficient and easy to deliver fluid to the perivenous space. It is also desirable to eliminate the need for multiple operators and multiple syringe change outs. It is also desirable to provide for user-controlled infusion volumes at consistent pressure levels and provide an infusion mechanism that is easy to handle and control. In addition, it is desirable to provide such a device that is inexpensive to manufacture, and is easy and inexpensive to use.

### SUMMARY OF THE DISCLOSURE

According to the principles of the present invention, a tumescent fluid infusion apparatus for use in treatment of a vascular disease is provided. The apparatus includes a needle having a channel and operable to penetrate a skin. A valve device is attached to the needle and is adapted to receive tumescent fluid from a fluid source. When the valve device is in an open position, it administers the tumescent fluid from the fluid source through the needle channel.

In another aspect of the invention, the valve device of the tumescent fluid infusion apparatus is in a normally closed position to block the administration of the tumescent fluid.

In another aspect of the invention, the valve device includes a manually depressible member that switches the closed position to an open position upon depression of the manually depressible member.

In another aspect of the invention, the fluid source is a pressurized source and the open position of the valve device allows the tumescent fluid to flow from the pressurized source through the needle channel.

In another aspect of the invention, release of the manually depressible member automatically closes the valve device to block the fluid flow.

In another aspect of the invention, the apparatus includes a flexible tube having one end coupled to the valve device and the other end adapted to be coupled to the fluid source.

In another aspect of the invention, the manually depressible member adjusts the flow rate of the fluid through the needle channel based on the amount of depression of the manually depressible member.

Advantageously, the present invention allows a single hand of a user to control the needle insertion and the infusion of the tumescent fluid to free the other hand for operating a probe such as an ultrasound probe to eliminate the requirement of a second operator. The infusion apparatus also allows a user to control the amount of infused fluid without requiring syringe changes.

### BRIEF DESCRIPTION OF THE DRAWINGS

FIG. 1 is a plan view of the endovascular tumescent injection technique of prior art.

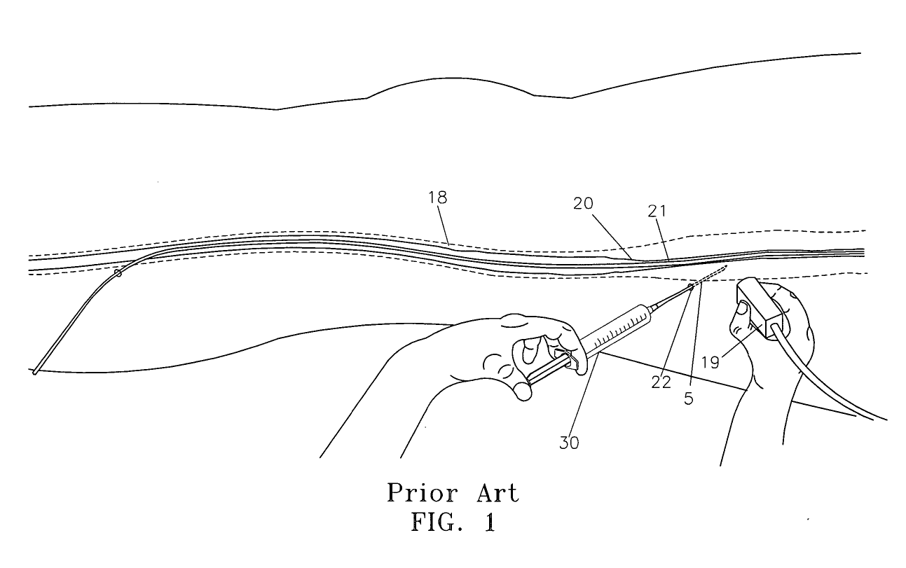

FIG. 2 is a plan view of an alternative endovascular tumescent injection technique of prior art.

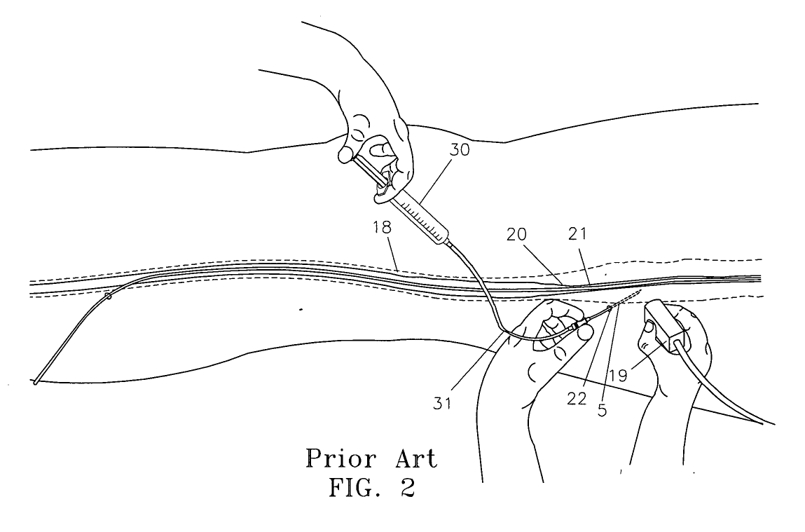

FIG. 3 is a plan view of an endovascular tumescent infusion apparatus according to the present invention.

FIG. 4 is a plan view of the endovascular tumescent infusion apparatus connected to a pressurized fluid reservoir.

FIG. 5A is a cross-sectional view of the endovascular tumescent infusion apparatus with the button valve in the closed position.

FIG. 5B is a cross-sectional view of the endovascular tumescent infusion apparatus with the button valve in the open position.

FIG. 6A and FIG. 6B are schematics of the endovascular tumescent infusion method of the present invention using the apparatus of FIG. 3.

### DETAILED DESCRIPTION OF THE INVENTION

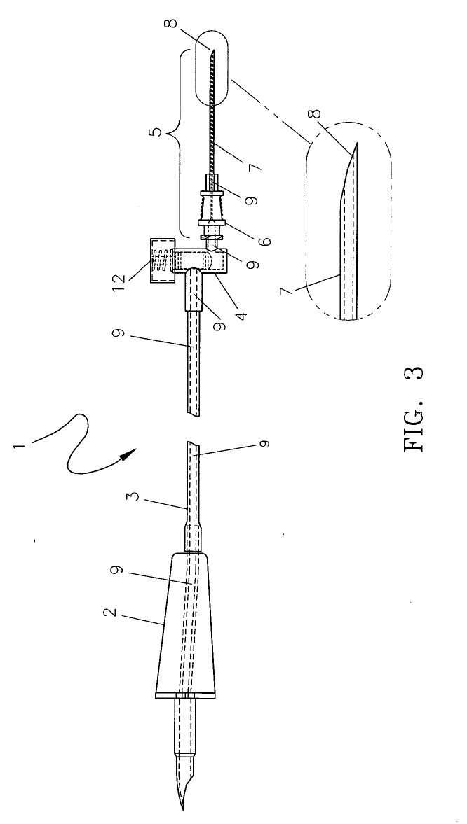

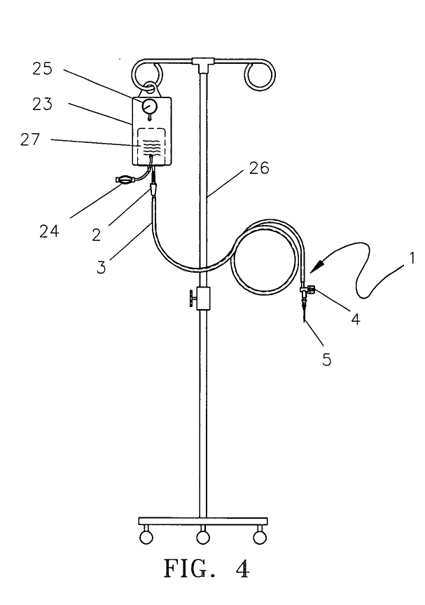

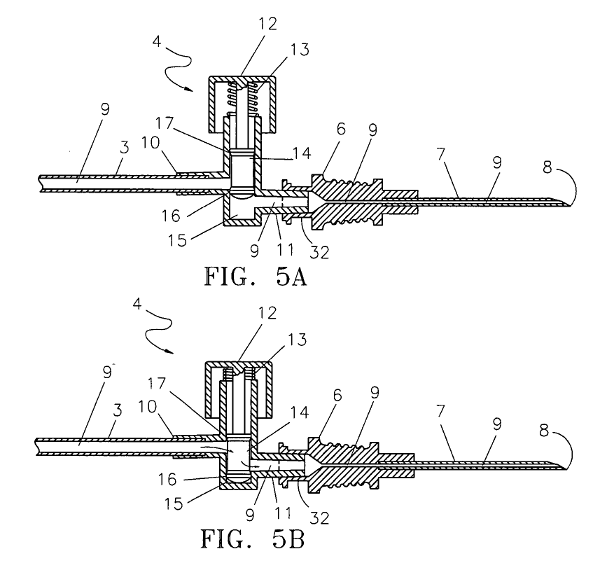

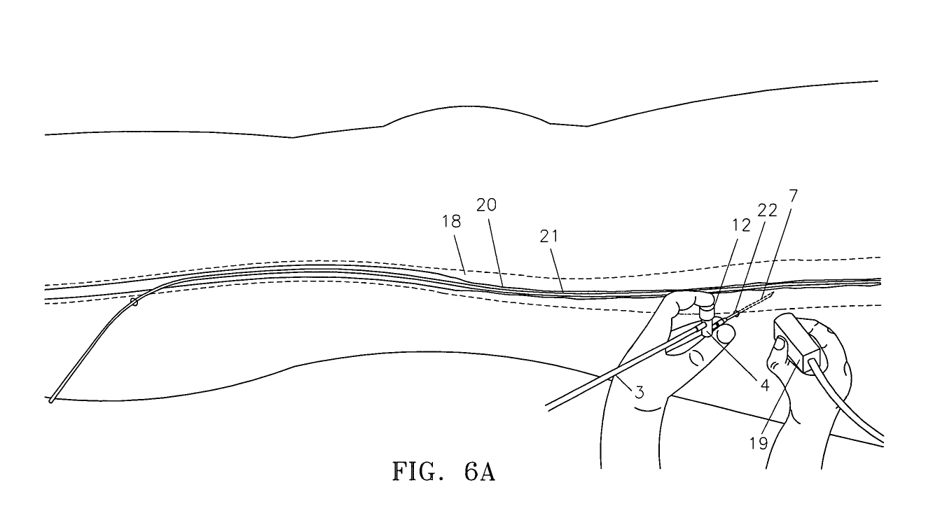

One embodiment of the present invention is shown in FIG. 3. The endovascular tumescent infusion apparatus 1 includes a non-vented spike 2 for connection to a fluid source such as a reservoir 27 (see FIG. 4), a flexible PVC tube or other similar type tubing 3, a button valve device 4 and a small gauge needle device 5. The needle device 5 is comprised of a needle 7 and a needle hub 6 having a needle hub port 32 (see FIGS. 5A and 5B). The needle 7 has a beveled needle tip 8. A fluid channel 9 extends from the spike 2 through the tubing 3, the lumen of the button valve 5, and the needle device 5.

The non-vented spike 2 is connected to a pressurized fluid reservoir 27 containing the tumescent fluid such as a lidocaine/saline mixture, as shown in FIG. 4. Pressurizing the reservoir 27 is accomplished by wrapping a standard pressure cuff 23 around the reservoir 27. Other alternative methods well known in the art may also be used to create a pressurized reservoir. Typically a 100 to 250 cc saline bag, commonly available, is used for the fluid reservoir. Lidocaine is injected into the saline bag through a port and then the solution is mixed. The spike 2 is inserted into the bag port to create a pressurized fluid connection/line between the reservoir 27 and the tumescent infusion apparatus 1. The reservoir 27 is pressurized by squeezing the bulb 24 which causes the cuff 23 to inflate, generating pressure against the fluid reservoir 27. Pressure levels of up to 300 mm/Hg may be used to ensure sufficient fluid flow into the perivenous space. The pressure dial 25 provides an indication as to pressure levels.

When the spike 2 is connected to the pressurized reservoir 27, a fluid channel 9 is created through the spike, the tubing 3 and the button valve device 4, at which point the fluid channel 9 is blocked by the normally closed position of the button valve device 4. Button valves by themselves, also known as trumpet valves, are well known in the prior art. The valve component illustrated within this application is disclosed, for example, in U.S. Pat. No. 5,228,646, which is incorporated herein by reference. Any valve device, especially one having a normally closed position, can be used in conjunction with this device.

Referring to 5A and 5B, the button valve device 4 includes an inlet port 10 connectable to the flexible tube 3, and an outlet port 11 connectable to the hub 6. Inlet port 10 is sealably connected to the tubing 3. Outlet port 11 allows for removable connection to the needle hub 6. The valve device 4 also includes a manually actuatable member such as a cap 12, return spring 13 that biases the valve device into a closed position, a plunger shaft 14 with a distal plunger seal 16 and a proximal plunger seal 17. In the embodiment shown, the manually actuatable member 12 is a manually depressible member that opens the valve device 4 by manual depression by the user. While the valve device 4 is shown with a manually depressible member 12, other types of valve actuating members can be used. For example, the present invention can be used with a roller switch that closes or opens in response to rotational movement of a rolling member or a sliding valve switch that uses a sliding member.

When in the normally closed position, the position of the plunger shaft 14 and the distal plunger seal 16 effectively blocks the passage of fluid through channel 9. Depressing the cap 12 compresses the return spring 13 causing the plunger shaft 14 to move deeper within the plunger cavity 15, as shown in FIG. 5B. When the plunger shaft 14 is repositioned as such, the distal plunger seal 16 no longer seals the channel 9 between the inlet port 10 and the outlet port 11. In the embodiment shown, the valve device 4 opens by manual pressure applied to the cap 12 along an axis which is substantially perpendicular to the longitudinal axis of the needle 9. The perpendicular axis of the cap movement allows a physician to more consistently maintain the depth of the inserted needle 7. Pressurized fluid then flows around the plunger shaft 14 as indicated by the arrows in FIG. 5B through channel 9 into the needle hub port 32. When the cap 12 is released, the return spring 13 expands to cause the plunger shaft 14 to return to the originally closed position as illustrated in FIG. 5A.

Alternatively, the valve opening of the valve device 4 can be made to vary according to the amount of depression of the manually depressible member 12 such that the flow rate of the fluid through the needle channel increases as the amount of depression of the depressible member 12 increases.

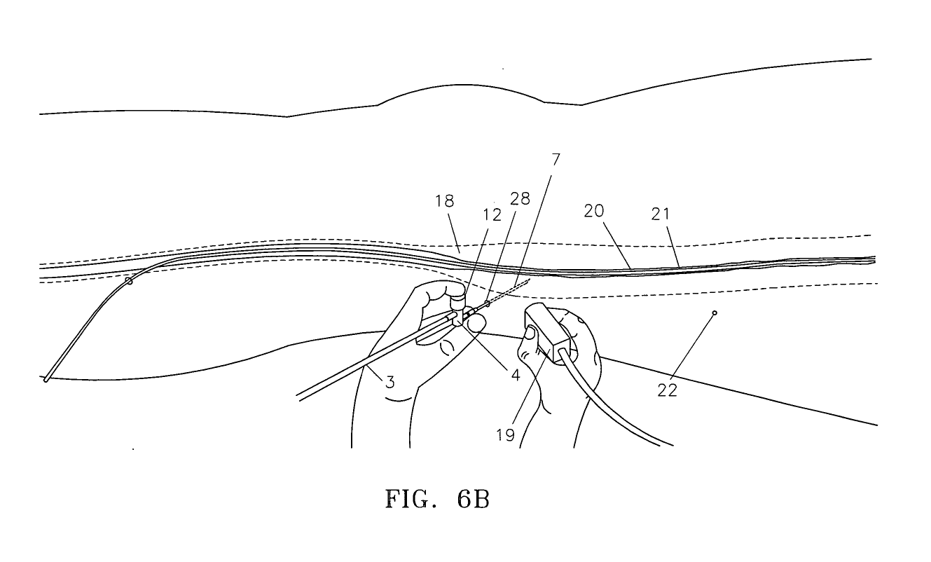

A preferred method of using the endovascular tumescent infusion apparatus 1 for treating a vascular disease will now be described with reference to FIG. 6A and FIG. 6B. The treatment procedure begins with the standard pre-operative preparation of the patient as is well known in the art. Prior to the procedure, the patient's diseased venous segments are marked on the skin surface. Typically, ultrasound guidance is used to map the greater saphenous vein 20 from the sapheno-femoral junction to the popliteal area.

The greater saphenous vein 20 is accessed using a standard Seldinger technique. A small gauge needle is used to puncture the skin and access the vein. A guide wire is advanced into the vein through the lumen of the needle. The needle is then removed leaving the guidewire in place. A hemostasis introducer sheath may be introduced into the vein over the guidewire and advanced to 1 to 2 centimeters below the sapheno-femoral junction. The distal end of a thermal treatment device such as a laser treatment device 21 is then inserted into and is advanced through the sheath. Alternatively, the thermal treatment device 21 may be inserted and advanced through the vein 20 without the use of a sheath.

Once the device is positioned correctly within the vein 20, the tissue immediately surrounding the diseased vessel segment is treated with percutaneous infusions of a tumescent anesthetic agent. In some cases, however, injection of tumescent anesthesia is done after the sheath placement but before the fiber introduction. As shown in FIG. 6A, the user inserts the needle 7 through the skin at puncture site 22 and into the perivenous space 18. The ultrasonic probe 19 is placed on the skin in the proximity of puncture 22 to provide an image of the needle 7 position in the perivenous space 18. One hand is used to position the needle device 5 while the other hand positions the ultrasonic probe 19.

To infuse the tumescent fluid, the user simply holds the needle device 5 with one hand and uses a finger to depress the cap 12 of the button valve device 4 in order to initiate fluid flow. As can be appreciated, this arrangement advantageously allows the user to maintain control of the needle device 5 and the infusion with one hand, freeing the other hand to position the ultrasound transducer 19. The user can easily control the infusion volume by holding the cap 12 down until the desired volume has been administered and then simply releasing the cap 12. Once the cap 12 is released, the valve device 4 automatically returns to a closed position as shown in FIG. 5A, preventing any further infusion of fluid.

When the tumescent fluid begins to dissipate radially into the surrounding tissue and is no longer flowing longitudinally within the perivenous space 18, as shown under the ultrasonic image, the user removes the needle 7 from puncture site 22. The needle is repositioned in another location, typically a few centimeters away from the original puncture site 22. The ultrasound transducer is also repositioned near the new needle location. FIG. 6B depicts the location of the repositioned needle 7 and transducer 19 at the second puncture site 28. Once correctly positioned and sufficiently imaged, the user infuses through the second puncture site by pressing down on the cap 12. The infused fluid anatomically isolates the vein from the surrounding structures by compressing the vein and creating a fluid barrier between the vein and surrounding tissue as shown in FIG. 6B.

Alternatively, other veins such as lesser saphenous veins may be targeted using alternative access techniques such as cut-down.

The entire length of the diseased vein segment is treated in this manner. Typically between 5 and 15 separate infusions are administered to sufficiently anesthetize the area and create a sufficient fluid barrier for treatment. A total of between 60 and 120 cc of fluid will be infused along the vein during treatment preparation. Once the vein has been sufficiently anesthetized, thermal energy is applied to the interior of the diseased vein. The thermal delivery system is slowly withdrawn through the vein until the entire vein segment has been treated.

The invention disclosed herein has numerous advantages over prior art treatment devices and methods. The current invention provides user control over the infused volume by allowing the user to monitor the infusion in real-time using the ultrasonic probe, providing improved visualization of the fluid flow.

Providing an infusion device which can be operated using a single hand, allows a single user to be able to simultaneously control the infusion and monitor the process real-time using an ultrasound probe. Thus, the present invention eliminates the need for two operators within the sterile field during the preparation of the vein.

Eliminating the need for multiple, small syringes results in a preparation procedure that is faster, easier and more precise. Preparing for infusion fluid with the current invention requires only a single connection to the reservoir and a step of pressurizing the reservoir. In addition, the risk of introducing air into the body through the exposed needle hub during syringe exchanges is eliminated with the apparatus and method of the current invention.

Accordingly, the advantages of the present endovascular tumescent infusion device include decreased procedural preparation time, convenience to the user and increased control over the infusion process. In addition, because common medical device components are used to assemble the device, it can be manufactured easily and at a low cost. Thus the device provides an inexpensive option for users in injecting tumescent fluid into the body.

The above description and the figures disclose particular embodiments of an endovascular tumescent infusion devices and method of treatment. It should be noted that various modifications to the device and method might be made without departing from the scope of the invention. The tumescent infusion needle and valve components can be of various designs as long as they provide ease of entry and controlled infusion volumes. For example, the valve can be of a configuration that allows for user control over flow rate. The needle configuration can be longer to provide fluid delivery further along the perivenous pathway. It may also be of a curved configuration to allow angled entry with longitudinal positioning adjacent to the vein. Coaxial needle configurations are also possible.

The technique for pressurizing the fluid reservoir can also be accomplished using other methods well known in prior art. The method of treatment can also be altered without departing from the scope of the invention. For example, the user may infuse longer perivenous space segments by increasing pressure levels. Veins other than the greater saphenous vein can be treated using the method described herein. Tumescent fluid injection for even non-venous structures is also possible using the present invention.

The foregoing specific embodiments represent just some of the ways of practicing the present invention. Many other embodiments are possible within the spirit of the invention. Accordingly, the scope of the invention is not limited to the foregoing specification, but instead is given by the appended claims along with their full range of equivalents.

1. A tumescent fluid infusion apparatus for use in treatment of a vascular disease, comprising:
a needle having a channel and operable to penetrate a skin; and
a valve device attached to the needle and adapted to receive tumescent fluid from a fluid source, the valve device in an open position being operable to administer the tumescent fluid from the fluid source through the needle channel.

2. The tumescent fluid infusion apparatus according to claim 1, wherein the valve device is in a normally closed position to block the administration of the tumescent fluid.

3. The tumescent fluid infusion apparatus according to claim 2, wherein the valve device includes a manually actuatable member that switches the closed position to the open position upon actuation.

4. The tumescent fluid infusion apparatus according to claim 3, wherein the manually actuatable member is a manually depressible member that switches the closed position to the open position upon depression of the manually depressible member.

5. The tumescent fluid infusion apparatus according to claim 1, wherein the valve device receives the tumescent fluid from a pressurized source and the open position of the valve device allows the tumescent fluid to flow from the pressurized source through the needle channel.

6. The tumescent fluid infusion apparatus according to claim 5, wherein the valve device includes a manually actuatable member that switches the closed position to the open position upon actuation.

7. The tumescent fluid infusion apparatus according to claim 6, wherein the manually actuatable member is a manually depressible member and release of the manually depressible member automatically closes the valve device to block the fluid flow.

8. The tumescent fluid infusion apparatus according to claim 1, further comprising a flexible tube having one end coupled to the valve device and the other end adapted to be coupled to the fluid source.

9. The tumescent fluid infusion apparatus according to claim 8, wherein the valve device includes a manually actuatable member that switches the closed position to the open position upon actuation.

10. The tumescent fluid infusion apparatus according to claim 9 wherein the manually actuatable member is a manually depressible member that adjusts the flow rate of the fluid through the needle channel based on the amount of depression of the manually depressible member.

11. A tumescent fluid infusion apparatus for use in thermal treatment of a vascular disease, comprising:
a needle having a channel and operable to penetrate into a perivenous space under the skin;
a needle hub having a channel in fluid communication with the needle channel;
a valve device being in a normally closed position and having an inlet connectable to a pressurized fluid source and an outlet attached to the needle hub, the valve device administering the tumescent fluid from the fluid source to the perivenous space through the needle channel when the valve device is changed to the open position.

12. The tumescent fluid infusion apparatus according to claim 11, wherein the valve device includes a manually actuatable member that switches the closed position to the open position upon actuation.

13. The tumescent fluid infusion apparatus according to claim 12, wherein the manually actuatable member is a manually depressible member that switches the closed position to the open position upon depression of the manually depressible member.

14. The tumescent fluid infusion apparatus according to claim 13, wherein release of the manually depressible member automatically closes the valve device to block the fluid flow.

15. The tumescent fluid infusion apparatus according to claim 11, wherein the valve device includes a manually actuatable member that switches the closed position to the open position upon actuation.

16. The tumescent fluid infusion apparatus according to claim 15, wherein the manually actuatable member is a manually depressible member that adjusts the flow rate of the fluid through the needle channel based on the amount of depression of the manually depressible member.

17. A tumescent fluid infusion apparatus for use in treatment of a vascular disease, comprising:
a pressurized fluid line;
a needle having a channel in fluid communication with the pressurized fluid line for fluid administration, the needle channel having a first axis; and
a valve device being coupled to the needle channel and having a manually operable member that opens and closes the needle channel from the pressurized fluid line, the manually operable member being biased to a normally closed position and being opened by manual pressure along a second axis substantially perpendicular to the first axis of the needle channel.

18. The tumescent fluid infusion apparatus according to claim 17, wherein the valve device is positioned adjacent to the needle to facilitate a single handed fluid infusion operation.

19. A method of treating a vascular disease, comprising:
inserting a needle of an infusion apparatus into the tissue and near a vessel to be treated, the infusion apparatus having a valve device attached to the needle and adapted to receive tumescent fluid from a pressurized fluid source, the valve device having a manually actuatable member in a normally closed position;
actuating the manually actuatable member to open the valve device while holding the inserted needle in place such that the tumescent fluid flows from the pressurized fluid source through the channel of the inserted needle, the actuation of the manually depressible member and the holding of the needle being performed using a single hand of a user.

20. The method according to claim 19, further comprising holding a probe near the vessel to be treated to monitor the infusion of the tumescent fluid, wherein the step of holding is performed using the other hand of the user.
